(19) United States
(12) Plant Patent Application Publication
(10) Pub. No.: US 2016/0120083 P1
(43) Pub. Date: Apr. 28, 2016

(54) Sweet orange tree named 'OLL-4'

(50) ;

(71) Applicant: Florida Foundation Seed Producers, Inc., Marianna, FL (US)

(72) Inventor: Jude W. Grosser, Winter Haven, FL (US)

(21) Appl. No.: 14/121,859

(22) Filed: Oct. 27, 2014

(51) Int. Cl. A01H 5/00

(52) U.S. Cl. PLT/202

(57) ABSTRACT

A new and distinct sweet orange clone, ‘OLL-4’ was produced via embryonic callus (tissue culture) from an original tree selection (‘OLL’) from an abandoned USDA field trial. ‘OLL-4’ produces easy-to-peel, round oranges with excellent internal and external color. Trees of ‘OLL-4’ exhibit improved cold and drought tolerance over industry standard oranges, and do not seem to exhibit alternate bearing.

### LATIN NAME OF THE GENUS AND SPECIES OF THE PLANT CLAIMED

Citrus sinensis

### VARIETY DENOMINATION

‘OLL-4’

### BACKGROUND OF THE INVENTION

The present invention relates to a new and distinct variety of sweet orange tree named ‘OLL-4’. ‘OLL-4’ is a somaclone regenerated from embryogenic callus (tissue culture) of ‘OLL’ (‘Orie Lee Late’) sweet orange, with high-quality fruit that usually matures in the standard ‘Valencia’ time period. ‘OLL’ (unpatented) is believed to be an irradiation-induced mutant line originating from ‘Pineapple’ sweet orange in an abandoned USDA trial attempting to generate a seedless ‘Pineapple’ orange. ‘OLL’ attracted attention because it never dropped fruit, a serious problem with ‘Pineapple’, and held quality late into the summer. Propagations of ‘OLL’ were not uniform and included some trees of poor growth and productivity, as well as robust high-yielding trees. In efforts to generate genetically stable clones from the ‘OLL’ selection, tissue cultures (embryogenic callus) were established for the creation of somaclones. At present, ‘OLL-4’ has been the highest yielding somaclone, and is the only remaining somaclone showing no Huanglongbing (HLB) disease symptoms. Trees of ‘OLL-4’ sweet orange topworked onto HLB-infected root/scaffold trees (‘Swingle’ and ‘Carrizo’) are also growing off quite well, but it is too early to determine the actual level of HLB tolerance. Topworked trees demonstrated true-to-typeness, as well as new trees grafted to experimental rootstocks also planted at St. Cloud, Fla.

### SUMMARY OF THE INVENTION

‘OLL-4’ produces round oranges with internal and external color similar (or better) to that of ‘Rhode Red Valencia’ (Table 1). Fruit of ‘OLL-4’ holds on the tree exceptionally well, and maintains quality into the summer. Fruit of ‘OLL-4’, like all of the ‘OLL’ oranges, peel easier than ‘Valencia’, which, combined with the attractive external color, provides excellent crossover potential as fresh fruit, thereby expanding the available fresh orange portfolio for Florida. ‘OLL’ seedling trees (including ‘OLL-4’) have shown good drought tolerance in the greenhouse, as overgrown trees in small pots rarely wilt compared to other sweet orange seedlings. This apparent improved stress tolerance may be useful against HLB disease. Limited yield data is available, but the original tree of ‘OLL-4’ has been the highest yielding tree among the ‘OLL’ somaclones over the past 3 years, producing more than five 90-lb. boxes of fruit per year, with a maximum of 6.13 boxes in 2014 (Table 1). Trees on ‘Swingle’ citrumelo appear to yield better than standard ‘Valencia’. Production of lbs solids on ‘OLL-4’ is not the highest among the ‘OLL’ somaclones (Table 2), but has been as good as or better than traditional ‘Valencia’. ‘OLL’ oranges, including ‘OLL-4’, have not exhibited the undesirable low ratios associated with ‘Valencia’ during the past two seasons (Tables 2 and 3). Alternate bearing has not been an issue on ‘OLL-4’. Sensory (taste panel) analyses have indicated that ‘OLL-4’ has a favorable ‘Valencia’-like flavor profile (Table 3). Top-worked and grafted trees of ‘OLL-4’ on various rootstocks are growing off quite well.

TABLE 1
Yield data from original ‘OLL’ somaclone trees on ‘Swingle’
citrumelo planted in 2001 (90 lb. boxes)

| | 2012 | 2013 | 2014 | TOTAL | AVG. |
|---|---|---|---|---|---|
| | BOXES | BOXES | BOXES | BOXES | BOXES |
| OLL-1 | 4.00 | 4.75 | 3.88 | 12.63 | 4.21 |
| OLL-2 | 3.00 | 3.75 | 2.34 | 9.09 | 3.03 |
| OLL-3 | 2.75 | 4.75 | 4.13 | 11.63 | 3.88 |
| OLL-4 | 5.75 | 5.25 | 6.13 | 17.13 | 5.71 |
| OLL-5 | 4.75 | 3.75 | 3.38 | 11.88 | 3.96 |
| OLL-6 | 2.75 | 2.75 | 5.88 | 11.38 | 3.79 |
| OLL-7 | 2.75 | 4.25 | 3.88 | 10.88 | 3.63 |
| OLL-8 | 1.75 | 0.00 | 0.00 | 1.75 | 0.58 |
| OLL-9 | 4.25 | 4.00 | 3.13 | 11.38 | 3.79 |
| OLL-10 | 5.00 | 5.75 | 5.38 | 16.13 | 5.38 |
| OLL-11 | 1.00 | 1.50 | 1.38 | 3.88 | 1.29 |
| OLL-15 | 2.00 | 2.75 | 2.88 | 7.63 | 2.54 |
| OLL-16 | 3.00 | 3.75 | 2.63 | 9.38 | 3.13 |
| OLL-19 | 4.00 | 5.00 | 3.88 | 12.88 | 4.29 |
| OLL-20 | 2.00 | 5.25 | 4.13 | 11.38 | 3.79 |
| OLL-21 | 3.75 | 4.75 | 4.13 | 12.63 | 4.21 |
| OLL-22 | 5.25 | 4.50 | 3.38 | 13.13 | 4.38 |
| OLL-23 | 3.75 | 6.75 | 3.88 | 14.38 | 4.79 |
| OLL-25 | 3.25 | 3.75 | 2.38 | 9.38 | 3.13 |
| OLL-27 | 4.25 | 4.50 | 5.38 | 14.13 | 4.71 |

TABLE 2
Juice Quality Data from ‘OLL’-Series
Somaclones (data from Mar. 02, 2012).

| | Lbs. Juice | | Total | | Lbs. Solids | |
|---|---|---|---|---|---|---|
| Variety | Per Box | Acid | Brix | Ratio | Per Box | Color |
| ‘Valencia’ | 54.41 | 1.00 | 12.03 | 12.03 | 6.55 | 38.60 |
| ‘OLL 20’ | 53.23 | 0.84 | 12.10 | 14.40 | 6.44 | 40.50 |
| ‘OLL 27’ | 54.99 | 0.84 | 12.34 | 14.69 | 6.79 | 39.80 |
| ‘OLL 25’ | 57.58 | 0.79 | 12.81 | 16.22 | 7.38 | 40.20 |
| ‘OLL 9’ | 56.30 | 0.94 | 12.97 | 13.80 | 7.30 | 39.90 |
| ‘OLL 15’ | 49.48 | 0.99 | 14.56 | 14.71 | 7.20 | 39.50 |
| ‘OLL 2’ | 51.55 | 0.77 | 12.77 | 16.58 | 6.58 | 41.00 |
| ‘OLL 3’ | 54.52 | 0.98 | 12.92 | 13.18 | 7.04 | 40.30 |
| ‘OLL 4’ | 55.52 | 0.92 | 12.60 | 13.70 | 7.00 | 40.70 |
| ‘OLL 7’ | 57.51 | 0.94 | 13.20 | 14.04 | 7.59 | 41.20 |
| ‘Swingle’ Control | 48.46 | 0.79 | 12.91 | 16.34 | 6.26 | 39.10 |
| ‘OLL 21’ | 58.20 | 0.77 | 12.38 | 16.08 | 7.20 | 40.30 |
| ‘OLL 10’ | 55.55 | 0.82 | 12.42 | 15.15 | 6.90 | 40.60 |
| ‘OLL 5’ | 53.92 | 0.93 | 13.13 | 14.12 | 7.08 | 40.10 |
| ‘OLL 23’ | 57.21 | 0.81 | 12.12 | 14.96 | 6.93 | 39.90 |
| ‘OLL 19’ | 54.02 | 0.84 | 12.32 | 14.67 | 6.65 | 40.90 |
| ‘OLL 1’ | 55.10 | 0.90 | 12.90 | 14.33 | 7.11 | 41.50 |
| ‘OLL 3-9’ | 58.19 | 0.96 | 12.85 | 13.39 | 7.48 | 40.80 |
| ‘OLL 3-10’ dwarf | 53.80 | 0.80 | 12.51 | 15.64 | 6.73 | 41.20 |
| ‘OLL 8’ | 56.43 | 0.77 | 11.99 | 15.57 | 6.77 | 40.70 |

TABLE 3
Sensory analysis of Not From Concentrate (NFC) juice from OLL oranges and ‘Valencia’ control.

| | | | | | | | | | | | Avg. |
|---|---|---|---|---|---|---|---|---|---|---|---|
| | | | | | | | | | | | Overall |
| ANALYSIS | SAMPLE | BRIX | ACID | | | | OIL | SINKING | VIT. C | Limonin | Flavor |
| DATE | ID | COR. | % | RATIO | pH | COLOR | % | PULP | (mg/100 ml) | ppm | Score |
| Mar. 21, 2014 | OLL # 10 | 12.97 | 0.88 | 14.74 | 3.78 | 39.53 | 0.016 | 14.0 | 53.79 | 2.5 | 3.33 |
| Mar. 21, 2014 | OLL # 11 | 12.37 | 0.85 | 14.55 | 3.77 | 40.50 | 0.012 | 12.0 | 45.27 | 2.1 | 3.25 |
| Mar. 21, 2014 | OLL # 27 | 12.53 | 0.87 | 14.41 | 3.77 | 40.62 | 0.012 | 12.0 | 42.65 | 1.1 | 3.25 |
| Mar. 21, 2014 | OLL # 8 | 13.26 | 0.97 | 13.67 | 3.75 | 39.30 | 0.014 | 12.0 | 52.55 | 2.6 | 3.33 |
| Mar. 21, 2014 | OLL # 2 | 12.09 | 0.81 | 14.93 | 3.85 | 40.50 | 0.012 | 12.0 | 44.24 | 1.9 | 3.33 |
| Mar. 21, 2014 | OLL # 20 | 12.42 | 0.89 | 13.96 | 3.77 | 40.09 | 0.015 | 14.0 | 48.68 | 1.3 | 3.25 |
| Mar. 21, 2014 | OLL # 7 | 12.71 | 0.85 | 14.95 | 3.83 | 40.81 | 0.013 | 12.0 | 38.54 | 1.3 | 3.25 |
| Mar. 21, 2014 | OLL # 6 | 12.10 | 0.87 | 13.91 | 3.80 | 40.58 | 0.012 | 12.0 | 40.55 | 1.4 | 3.25 |
| Mar. 21, 2014 | OLL # 1 | 12.47 | 0.95 | 13.13 | 3.73 | 40.48 | 0.011 | 12.0 | 45.95 | 2.2 | 3.42 |
| Mar. 21, 2014 | OLL # 16 | 12.34 | 0.87 | 14.18 | 3.75 | 40.25 | 0.015 | 12.0 | 44.47 | 1.6 | 3.50 |
| Mar. 21, 2014 | OLL # 5 | 11.16 | 0.77 | 14.49 | 3.90 | 41.09 | 0.011 | 10.0 | 35.07 | 2.2 | 3.42 |
| Mar. 21, 2014 | OLL # 21 | 11.69 | 0.87 | 13.44 | 3.82 | 40.63 | 0.013 | 12.0 | 38.34 | 2.5 | 3.42 |
| Mar. 21, 2014 | OLL # 25 | 13.36 | 0.87 | 15.36 | 3.72 | 40.16 | 0.018 | 12.0 | 42.21 | 1.5 | 3.42 |
| Mar. 21, 2014 | OLL # 19 | 12.80 | 0.81 | 15.80 | 3.84 | 40.59 | 0.017 | 12.0 | 42.34 | 2.0 | 3.50 |
| Mar. 21, 2014 | OLL # 4 | 12.54 | 0.85 | 14.75 | 3.84 | 40.74 | 0.015 | 12.0 | 40.04 | 1.0 | 3.25 |
| Mar. 21, 2014 | N10-25 | 12.96 | 0.93 | 13.94 | 3.76 | 38.67 | 0.020 | 14.0 | 41.89 | 1.0 | 3.33 |
| Mar. 21, 2014 | OLL # 9 | 12.59 | 0.85 | 14.81 | 3.80 | 40.81 | 0.013 | 12.0 | 38.35 | 1.4 | 3.25 |
| Mar. 21, 2014 | Valencia | 12.07 | 0.95 | 12.71 | 3.69 | 38.45 | 0.012 | 10.0 | 40.35 | 0.8 | 3.42 |
| Mar. 21, 2014 | OLL # 23 | 11.55 | 0.76 | 15.20 | 3.88 | 40.69 | 0.012 | 10.0 | 34.54 | 0.9 | 3.25 |
| Mar. 21, 2014 | OLL # 3 | 12.37 | 0.91 | 13.59 | 3.72 | 40.12 | 0.015 | 12.0 | 40.30 | 3.0 | 3.58 |

### BRIEF DESCRIPTION OF THE DRAWINGS

‘OLL-4’ is illustrated by the accompanying photographs, which show the tree's form, foliage and fruit. The colors shown are as true as can be reasonably obtained by conventional photographic procedures. The photographs are of a tree approximately 13 years old.

FIG. 1—Shows the internal and external appearance of the fruit.

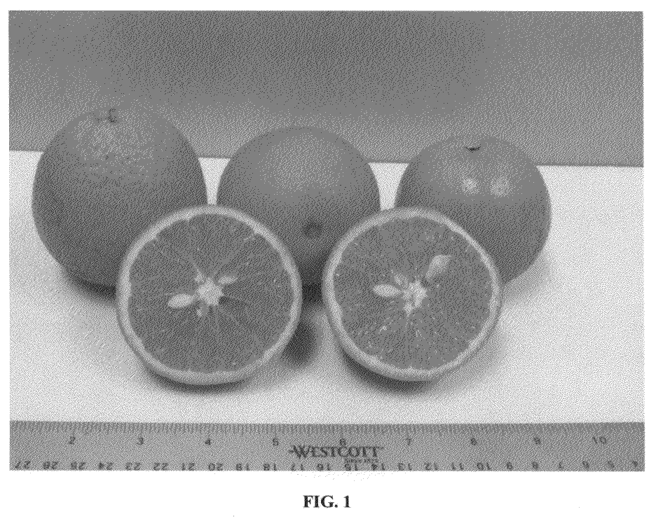

FIG. 2—Shows the overall mature plant growth habit in the spring time.

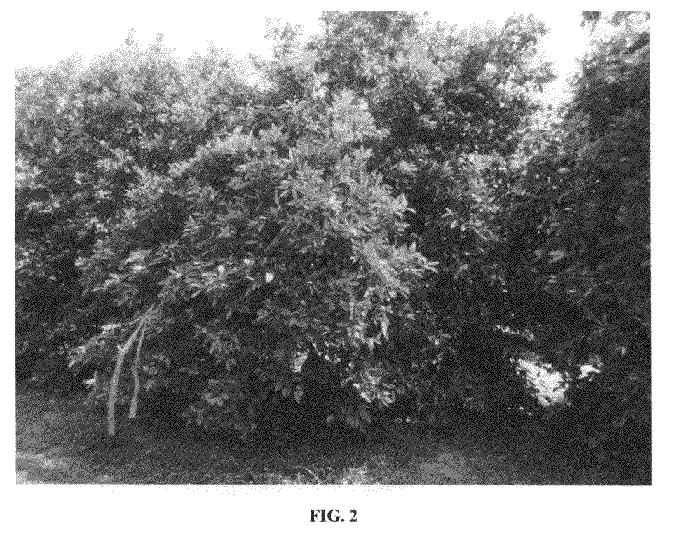

FIG. 3—Shows a close-up of leaves and a mature fruit.

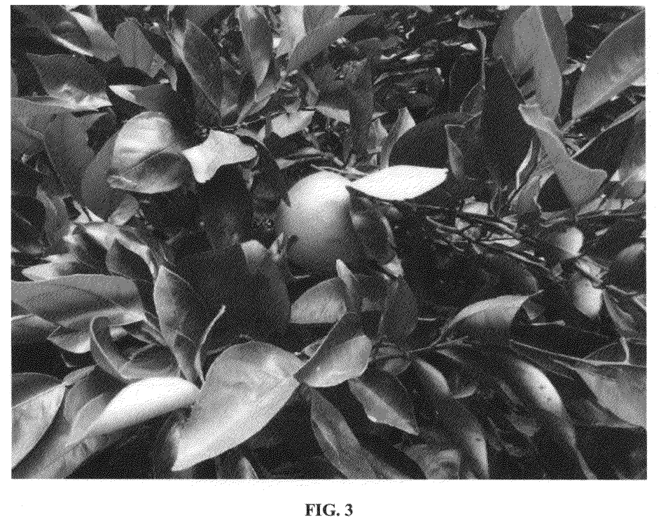

FIG. 4—Shows a close-up of the mature fruit.

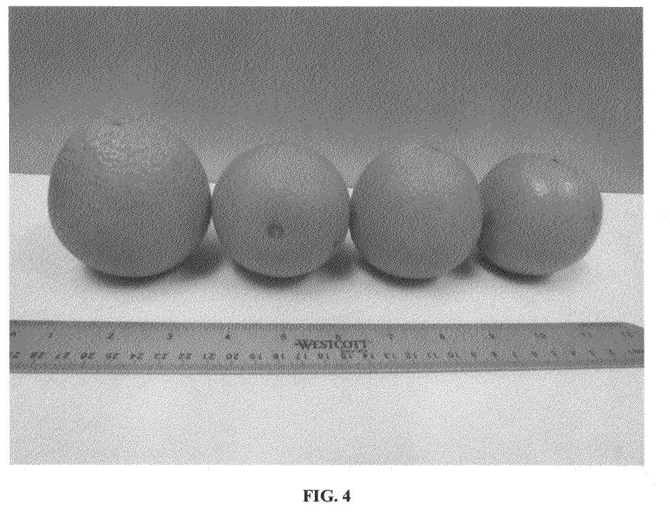

### DETAILED BOTANICAL DESCRIPTION

Phenotypic Description of Citrus sinensis ‘OLL-4’

The following detailed description sets forth the distinctive characteristics of ‘OLL-4’. The present botanical description is that of the variety grown on a 13-year-old tree growing on ‘Swingle’ citrumelo rootstock (C. paradisi×Poncirus trifoliata) at St. Cloud experimental block, and a top-worked tree at the same location. The colors (except those in common terms) are described from the R.H.S. Colour Chart published by The Royal Horticultural Society in London (second edition), in association with the Flower Council of Holland.
Classification:
   Botanical.—Citrus sinensis (putative).
   Common name.—Sweet orange.
Parentage:
   Female parent.—‘OLL’ (‘Orie Lee Late’) sweet orange tree (unpatented).
   Male parent.—N/A.
Tree:
   Ploidy.—Diploid.
   Size.—Medium large.
   Tree height.—5.8 m.
   Tree spread.—5.0 to 5.5 m.
   Vigor.—Vigorous.
   Density.—Canopies are quite dense.
   Form.—The tree is obloid shaped with both lateral and upright branches growing; branches with fruit exhibit drooping.
   Growth habit.—Both upright and lateral growth with low medium angle.
Trunk:
   Trunk diameter.—17.8 cm in diameter at 30 cm above the ground, 16-year-old tree.
   Trunk texture.—Smooth.
   Trunk bark color.—RHS 199C (greyed-brown); irregularly striated with RHS 189A (greyed-green).
Branches:
   Crotch angle.—First crotch forms a 60 to 65-degree angle, middle crotch forms a 35-degree angle.
   Branch length.—Branch reaches 5.6 meters from the first crotch to the tip of the branch.
   Branch texture.—Relatively smooth, occasionally with small thorns or spines.
   Branch color (shoots from previous flush, hardened, and 4 to 5 mm in diameter).—RHS 137A (green).
Leaves:
   Size (lamina average).—Length: 120 mm Width: 57.8 mm.
   L/W ratio.—2.1.
   Thickness.—Regular and average compared to commercial sweet orange hybrids.
   Type.—Simple.
   Shape.—Elliptical.
   Apex.—Retuse.
   Base.—Acute to sub-obtuse.
   Margin.—Entire and smooth undulate.
   Surface.—Upper surface: Glabrous Lower surface: Medium veins that are pinnately netted.
   Color.—Upper surface (adaxial): RHS 137A (green) Lower surface (abaxial): RHS 143C (green).
   Petiole.—Shape: Brevipetiolate (shorter than leaf lamina); junction between petiole and lamina is articulate Width (petiole wing): Narrow Shape (petiole wing): Obovate Length: 25.5 to 26 mm Width: 7.2 to 7.3 mm Color: RHS 137A (green).
Flowers and flower buds:
   Type.—Hermaphrodite.
   Bearing.—Flowers grow from leaf axillaries and leaf terminals in small clusters or as individuals. Each flower branch consists of 4-12 flowers.
   Flower diameter.—Fully open flower with an average diameter of 34.4 to 35 mm.
   Flower depth.—Typical flower has an average depth of 20 mm.
   Blooming period.—First bloom: Observed Mar. 20, 2014 Full bloom: Observed Mar. 28, 2014.
   Flower bud size.—Length: Initial visible flower bud is 3.5 mm in length; mature flower bud is 16.6 mm in length Diameter: Initial visible flower bud is 2.5 mm in diameter; mature flower bud is 7.1 mm in diameter.
   Flower bud shape.—Initial visible flower bud has a slightly elongated ball shape; mature flower has an elongated olive shape.
   Color.—RHS 145D (yellow-green) for initial visible flower bud; RHS NN155D (white) for mature flower bud with RHS 149D (yellow-green) spots distributed at the tip of the flower bud.
   Flower petals.—Number: 5 Length: 20 mm Width: 7.3 Shape: Flat, spatula shaped Apex shape: Smooth, acute shaped Base shape: Even obtuse.
   Color.—Upper surface: RHS NN155D (white) Lower surface: RHS NN155C (white) with RHS 149D (yellow-green) spots distributed toward to the petal apex Margin: Smooth.
   Sepal.—Number (per flower): 5 per flower Shape: Delta shaped with an acute angle at apex Length: 3.8 mm Width: 3.5 mm Apex shape: Triangle shaped Margin: Smooth.
   Color.—Upper surface: RHS 145D (yellow-green) Lower surface: RHS 145C (yellow-green).
   Pedicel.—Number: 1 Length: 6.7 to 6.8 mm Diameter: 1.4 to 1.5 mm Color: RHS 145B (yellow-green).
   Reproductive organs.—Fertility: Appears self-fertile Stamen length: 12.5 to 12.7 mm Anther Length: 3.4 mm Width: 1.1 to 1.2 mm Color: RHS 12C (yellow) Filament length: 10.2 to 10.3 mm Pollen amount: Abundant Pollen color (general): RHS 14A (yellow).
   Pistil.—Number: 1 Length: 12.0 to 12.8 mm Color: RHS 145C (yellow-green).
   Style length.—9.5 mm.
   Style diameter.—1.3 to 1.4 mm.
   Style color.—RHS 145C (yellow-green).
   Ovary shape.—Oval shaped.
   Ovary diameter.—2.3 mm.
   Ovary color.—RHS 145B (yellow-green).
   Fragrance.—Fragrant.
Fruit:
   Average weight (per individual fruit).—222.3 g.
   Size.—Uniform.
   Width.—74 to 77 mm on average.
   Length.—82.5 to 83.3 mm on average.
   Shape.—Earth shaped.
   Shape (cross-section).—Round.
   Apex.—Truncated.
   Apex cavity diameter.—N/A.
   Base.—No neck, with wrinkle at the shoulder area.
   Base cavity diameter.—6.3 to 6.4 mm.
   Harvesting.—First harvest can usually occur at the beginning of March (based on season and rootstock); last harvest can go into June, as fruit continue to hold on the tree for a long time with good quality.
   Fruit stem (short stem connecting the fruit).—Length: 10.8 mm Diameter: 3.8 mm Color: RHS 196A (greyed-green with RHS 137A (green) strip.
Skin:
   Adherence.—Adherence between albedo (mesocarp) and flesh (endocarp) is strong The adherence is evenly distributed from based to apex.
   Thickness.—4.4 to 4.5 mm on average.
   Texture.—Smooth.
   Color.—Flavedo (epicarp): Ranges between RHS 21A (yellow-orange) to RHS 21C (yellow-orange) Albedo (mesocarp): RHS 11D (yellow) Stylar end: Closed.
   Rind oil cell density.—208 oil cells/square cm.
Flesh:
   Number of segments.—Average between 10 and 11 segments per fruit.
   Segment walls.—Medium soft with sufficient strength to maintain integrity as separated.
   Juice.—Abundant.
   Color.—Uniformly RHS 24A (orange).
   Texture.—Medium soft.
   Vesicles.—Length: arranged from 16.3 to 16.9 mm on average.
   Diameter (thickness).—2.7 to 3.2 mm on average.
   Eating quality (tested at the beginning of March, varies from season to season).—Soluble solids (average): 12.6 Brix Acidity (average): 0.92% Sugar/Acid Ratio: 13.7.
Seeds:
   Type.—Mostly polyembryonic and occasionally monoembryonic.
   Number.—Ranges from 2 to 5; occasionally some fruit contains less than 2 seeds.
   Shape.—Seed shapes are not uniform; normal seeds are mostly ventricose/swollen shaped and clavate club shaped.
   Size.—Length: 15.4 to 15.5 mm Width: 8.9 to 9.0 mm.
   Seed coat color.—Outer Surface: RHS NN155A (white) and smooth Inner surface: RHS 165C (greyed-orange).
   Cotyledon color.—RHS 157D (green-white).
Resistance to disease: No obvious tree-debilitating disease problems have been observed in the trees or fruit of ‘OLL-4’ currently growing, but systematic resistance testing has not been performed. ‘OLL-4’ may be moderately susceptible to citrus canker, although canker has not been observed on ‘OLL-4’.

What is claimed is:

1. A new and distinct cultivar of sweet orange tree as illustrated and described herein.
